(19) United States
(12) Patent Application Publication
(10) Pub. No.: US 2004/0191344 A1
(43) Pub. Date: Sep. 30, 2004

(54) Pharmaceutical composition for prophylaxis or treatment of osteoporosis, and method to prepare the same

(76) Inventors: Hsiang Wang, Taipei (TW); Yu-Lin Mao, Taipei (TW)

Correspondence Address: Cynthia L. Pillote, Snell & Wilmer L.L.P., One Arizona Center, 400 East Van Buren, Phoenix, AZ, 85004-2202, US

(21) Appl. No.: 10/811,757

(22) Filed: Mar. 29, 2004

(30) Foreign Application Priority Data
Mar. 28, 2003 (TW) 92107128
Mar. 1, 2004 (TW) 93105332

(51) Int. Cl. A61K 35/78

(52) U.S. Cl. 424/776; 424/777

(57) ABSTRACT

A pharmaceutical composition for the prophylaxis and treatment of osteoporosis is provided. The composition is extracted from a mixture including Semen cuscutae, or Fructus cindii and Semen cuscutae, and then purified to obtain a large amount of active ingredients, such as coumarins and flavanoids. The pharmaceutical composition made from Semen cuscutae only is also effective. The aforesaid pharmaceutical composition can also be used with calcium and/or vitamin D₃.

### CROSS-REFERENCE TO RELATED APPLICATION

This Application claims priority to Taiwan Patent Application No. 92107128 filed on Mar. 28, 2003 and Taiwan Patent Application No. 93105332 filed on Mar. 01, 2004.

### FIELD OF INVENTION

The present invention relates to a pharmaceutical composition for the prophylaxis and treatment of osteoporosis and the method for preparation of the same, in particular, the invention relates to a composition for prophylaxis and treatment of osteoporosis with respect to the elderly and menopausal women and to a method of making the same.

### BACKGROUND OF THE INVENTION

Osteoporosis is a systemic disease that causes bone tissue loss, changes micro-structure of the bone, decreases bone tolerance of strength and increases the risk of bone fracture. Seniors and menopausal women are more susceptible to osteoporosis. The primary syndromes are systematic pain or pain in the lumbar and legs. As the disease progresses, deformation and fracture of the spine can occur under a slight external force. Deformation of the thoracic cage may further decrease lung capacity and impair cardiopulmonary functions.

As the improving living standard and long life make seniors an ever-increasing proportion of the population, the prophylaxis and treatment for osteoporosis have become an important and contiguous issue. The demands for a safe, effective and convenient modem formula (derived from traditional Chinese medicine) for the prophylaxis or treatment of osteoporosis may improve the living standard of the elderly and menopausal women.

As far as the prior art is concerned, treatments of osteoporosis can be categorized into three major types. The first type inhibits the expression of osteoclasts. Medications with ingredients such as estrogen, calcitonin, bisphosphates, ipriflavone and calcium fall into the first category. The second type facilitates the formation of osteoblasts, with active ingredients such as active vitamin D, fluoride, synthetic steroids and parathyroid hormone (PTH). Both types of treatments have some effect in terminating further bone loss and increasing bone mass. However, due to numerous side effects, long-term complications, high costs or dubious efficacy, these two types of treatments fall short of being ideal drugs for osteoporosis.

The third type is based on the traditional Chinese medicine theory. According to the theory from traditional Chinese medicine, osteoporosis is considered to be deeply associated with the so-called “Kidney Deficient” abstract state. The major symptoms are bone ache, pain in the waist and the back, and weakness in the loins and legs, which happen to be the same as those of osteoporosis. It is suggested that traditional Chinese medicine prevents and treats osteoporosis through systematic and multi-mode regulations of the bodily functions.

Also, modern medical science has discovered that the level of estrogen is related to the Yin-Yang balance of “Kidney” in traditional Chinese medicine. The Kidney-Reinforcing therapy may enhance immune system, restore the functions of pituitary gland—the target organ, promote the accumulation of bone calcium, inhibit the expression of osteoclasts and facilitate the formation of osteoblasts. Accordingly, it not only may slow down the progression of osteoporosis but also could possibly reverse the process.

The herbal medicine Fructus Cnidii is the dried ripe fruit of Cnidium monnieri (L) Cuss, which is an herb of fruit kind. The effective ingredients are osthole and imperatorin of the coumarins class. According to traditional Chinese medicine, Fructus cindii is effective in invigorating the kidneys, enhancing sexual capability, and preventing menopausal women from osteoporosis. Research shows that Fructus cindii and total coumarins are effective in the prophylaxis of osteoporosis in rats induced by ovariectomy or steroids. One of the major active ingredients, osthole, can rebalance the bone tissue conversion for menopause female, prevent bone loss and maintain the normal level of bone mass by inhibiting the expression of osteoclasts and facilitating the formation of osteoblasts.

Recently, many papers have been published on the clinical use of Fructus cindii compounds, but mainly as treatment for gynecological and dermatological diseases. In addition, the followings are some bone-related medical preparations with respect to Semen cuscutae:

China Patent Application No. 95109546.3, disclosed a medicine for treating bone hyperplasia, made from the processed powder of Radix Angelicae Sinensis, Rhizome Ligustici, Radix Paeoniae Alba, Radix Rehmanniae Praeparata, Cortex Eucommiae, Radix Dipsaci, Cortex Acanthopanacis Radicis, Rhizome Drynariae, Ramulus Cinnamomi, Radix Notoginseng, Radix Astragali seu Hedysari, Fructus Psoraleae, Semen Cuscutae, Radix Codonopsis Pilosulae, Fructus Chaenomelis, Artemisiae Anomalac Herba, Pyritum, Fructus Crataegi, Eupolyphaga sinensis Walker, leopard bones or dog bones, and Dendrobium parishii.

China Patent Application No. 96116013.6, disclosed a medicine for treating bone hyperplasia, made from Radix Rehmanniae, Cotex Lycii Radicis, Semen Cuscutae, Ramulus Wallichii seu puberulli, Radix Polygalae, Achyranthes bidentata, Gentiana crassicaulis, Caulis Spatholobi, Rhizoma Cyperi, Radix Glycyrrhizae, Rhizoma Dioscoreae, and Semen Coicis.

China Patent Application No. 96120912.7, disclosed a medicine for treating bone hyperplasia, which contains Radix Rehmanniae Praeparata, Fructus Psoraleae, Herba Epimedii, Semen Cuscutae, Cortex Acanthopanacis Radicis, Herba Cistanches, Male Bombyx mori, Cortex Eucommiae (processed with salt), Cibotium barometz, Fructus Schisandrae, Caulis Spatholobi, Radix Achyranthis Bidentae, Flos Carthami, dog's bone powder, scorpion, Radix Angelicae Sinensis, Radix Paeoniae Alba, Radix Glycyrrhizae, Panax Gingseng, Fruxtus Ziziphi Jujibae, Poria, Herba Cynomorii, Radix Notoginseng, and the powder mixture is further made by adding equivalent weight of Olibanum and Myrrha . . . , etc., being thoroughly powdered.

China Patent Application No. 98120495.3, disclosed a traditional Chinese medicine composition for treating bone hyperplasia, which contains Radix Astragali seu Hedysari, Radix Rehmanniae, Radix Codonopsis Pilosulae, radix Salviae Miltiorrhizae, Semen Cuscutae, Fructus Ligustri Lucidi, Radix Angelicae Sinensis, Rhizoma Sparaganii, Radix Paeoniae Rubra, Ramulus Mori, Radix Clematidis, Caulis Spatholobi, Herba Epimedii, Rhizoma seu Radix Notopterygii, Radix Dipsaci, Radix Puerariae, Radix Achyranthis Bidentatae, Cortex Eucommiae, Olibanum, Myrrha, Radix Aconiti Praeparata, Cortex Cinnamomi, Radix Ledebouriellae, Radix Glycyrrhizae, Caulis Piperis Futokadsurae, Scrophularia ningpoensis, Radix Rehmanniae Praeparata, Artemisia capillaries, Fruxtus Ziziphi Jujibae, Peng-goling.

China Patent Application No. 97112860.X, disclosed a health product for the prophylaxis/treatment of osteoporosis, made from bone powder, Radix Rehmanniae Praeparata, Rhizoma Dioscoreae, Fructus Corni, Fructus Lycii, Semen Cuscutae, Radix Angelicae Sinensis, Rhizome Alismatis, Poria, Pericarpium Citri reticulatae, Radix Codonopsis Pilosulae, Radix Astragali seu Hedysari, Citrus aurantium, Fructus Meliae Toosenddan, Radix Aucklandiae.

China Patent Application No. 00124955.X, disclosed a Chinese traditional medicine preparation for the treatment of femoral head necrosis, consisting of formulation I: Flos Carthami, Colla Corii Asini, Radix Rehmanniae Praeparata, Semen Persicae, Olibanum, Squama Mantitis, Radix Notoginseng, Herba Asari, Strychnos nuxvomica L., Radix Angelicae Sinensis, Semen Cuscutae, radix Salviae Miltiorrhizae, Achyranthes aspera, Radix Paeoniae Alba, Radix Astragali seu Hedysari, Radix Dipsaci, Fructus Chaenomelis, Lumbricus, scorpion, dead worm, Caulis Sinomenii, chilopod, Dendrobium parishii, and formulation II: ground beetle . . . , etc. and external use drug: charcoal, Phellodendron chinense, Pinellia ternata, Avicennia germinans, black chicken head, black chicken legs and claws, and white sugar.

China Patent Application No. 02120834.4, disclosed a traditional Chinese oral medication for the treatment of orthopaedic disease, consisting of turtle bone, Rhizome Drynariae, horn of cervus nippon temminck, Radix Rehmanniae Praeparata, Radix Notoginseng, Cortex Eucommiae, Lycium barbarum, Semen Cuscutae, ground beetle, Myrrha, scent, Radix Dipsaci, Daemonorpsdraco Blume, Talinum triangulare, Cornus officinalis.

China Patent Application No. 02119186.7, disclosed a medicine for the prophylaxis and treatment of bone fracture and osteoporosis, made from Veronica anagallis-aquatica, scent, Oryza sativa, Olibanum, Atractylodes macrocephala Koidz and Semen Cuscutae.

The oral acute LD₅₀ of the total coumarins in Fructus cindii is 2.44±0.05 g/kg (see Chinese Herbal Medicine, published by State Administration of Traditional Chinese Medicine, PROC, May 1999). The potential toxicity of Fructus cindii makes it extremely important to administer the dosage with precision.

Furthermore, several factors limit the applications in the prior art. (1) The preparations are usually in liquid form and not convenient to administer. (2) The complex mix of ingredients makes it difficult to standardize the preparations. (3) No modern administration method is available.

Considering the side effects, complications, expensive price and inconvenient administration found in the prior art, the demand is high for a product that is of minimal toxicity, clearly efficacious and easy to administer.

### SUMMARY OF THE INVENTION

One aspect of the present invention provides a pharmaceutical composition for the prophylaxis or treatment of osteoporosis and a method for preparation. The pharmaceutical composition contains traditional Chinese herbs, i.e. composite composition of mixed extract of Semen cuscutae and Fructus cnidii, as well as additive(s), such as one or more pharmacologically acceptable adjuvant and/or carrier and/or excipient. The combination can decrease the toxicity of Fructus cnidii, while maintaining the efficacy.

Another aspect of the present invention provides a pharmaceutical composition for the prophylaxis or treatment of osteoporosis, containing the extract of Semen cuscutae and additive(s) and its preparation method.

Still another aspect of the present invention provides a pharmaceutical composition for the prophylaxis or treatment of osteoporosis. In addition to Semen cuscutae, or Semen cuscutae and Fructus cnidii, the pharmaceutical composition further contains one or more calcium-containing substance(s) and/or vitamin D₃ to enhance the claimed efficacy in the prophylaxis or treatment of osteoporosis.

Yet another aspect of the present invention provides a method for preparing a pharmaceutical composition for osteoporosis prophylaxis and treatment, comprising the steps of

(a) providing a material comprising Semen cuscutae, or Semen cuscutae and Fructus cnidii;

(b) adding a sufficient extraction solvent into the material to obtain an extraction mixture, then leave the extraction mixture under heating;

(c) filtering the extraction mixture after the extraction mixture cools down;

(d) drying and pulverizing the extraction mixture to obtain an extract;

(e) dissolving the extract in a solvent and pouring the extract into a prepared porous resin column; and

(t) performing elution by adding solutions of different concentrations of ethanol and water as eluant into the prepared porous resin column to obtain the pharmaceutical composition.

Or, a method for preparing the pharmaceutical composition for the prophylaxis and treatment of osteoporosis, comprising the steps of:

(a) mixing a first amount of Fructus cindii with a second amount of Semen cuscutae to form a mixture;

(b) adding sufficient ethanol of concentration of about 20% to about 80% into the mixture then heating;

(c) filtering the mixture after the mixture cools down;

(d) drying and pulverizing the mixture to obtain an extract;

(e) dissolving the extract in a solvent and pouring the extract into a prepared porous resin column; and

(t) performing elution by in succession adding solutions of different concentrations of ethanol and water as eluant into the prepared porous resin column to obtain the pharmaceutical composition.

The pharmaceutical composition of the present invention is made from the mixture of the two Chinese traditional herbs, Semen cuscutae and Fructus cnidii. After extraction and purification, the mixture yields a pharmaceutical composition with a high percentage of effective ingredients, i.e. coumarins and flavnoids, which are universally recognized as botanical hormones and are gaining importance in the study of osteoporosis. The single extract of Semen cuscutae also contain botanical hormones such as coumarins and flavnoids.

The pharmaceutical composition of the present invention can substantially increase bone mineral density BMD and calcium and phosphorous levels in bone ash. In addition, unlike the separate administration of Fructus cnidii, the pharmaceutical composition of the present invention has little oral toxicity.

Yet another aspect of the present invention is the discovery that the extract from Semen cuscutae itself is also effective in the treatment of osteoporosis and has little oral toxicity. In the present invention, the extract of Semen cuscutae and Fructus cindii or the extract of Semen cuscutae alone can further be combined with calcium-containing substance(s) and/or vitamin D₃ to further enhance BMD and the calcium and the phosphorous levels in bone ash.

These and other objects, features, and advantages of the present invention will become more apparent from the following detailed description of illustrative embodiments thereof, which should be read in connection with the accompanying tables.

### DETAILED DESCRIPTION

According to one embodiment of the present invention, the pharmaceutical composition contains two traditional Chinese herbs, Semen cuscutae and Fructus cnidii. Semen cuscutae and Fructus cindii plants are abundant in nature and relatively inexpensive.

With higher concentration of the effective ingredients, it is easy to control the quality of the pharmaceutical composition.

The herb Semen cuscutae is the dried ripe seeds of Cuscuta chinensis Lam. of convolvulaceae, which is an herb of fruit kind. According to the inventors' research, the effective ingredients in the herb are quercetin and kaempferol in the family of flavnoids.

Semen cuscutae is pharmaceutically useful in essence of “liver” and “kidney,” treats dysfunction of corpus luteum and restores balance of the female reproductive endocrine system. Therefore, it is based on the traditional Chinese medicine theory that osteoporosis is related to “Kidney Deficiency” that the present invention use Semen cuscutae to enhance the efficacy of Fructus cnidii. The flavnoids contained in Semen cuscutae can act like estrogen to abstractly invigorate “the kidney”. In addition to enhancing the efficacy of Fructus cnidii, Semen cuscutae itself is also effective in treating osteoporosis.

The following non-limiting examples illustrate various compositions and methods in accordance with the present invention. These examples are merely illustrative, and it is not intended that the invention be limited to these illustrative examples.

### EXAMPLE 1

A 5L container contains a mixture of 250 g of Fructus cnidii, preferably in the form of powder, and 150 g of Semen cuscutae, preferably in the form of powder. Later, ethanol (or other polar solvent(s)) of about 4-10 times volume of the mixture of concentration deom about 20% to about 80%, preferably about 50% ethanol of 2800 ml as extraction solvent, is added thereto. The liquid mixture is heated to about 50-70° C. for about 3-10 hours. It is then cooled and filtered after cooling. Next, the filtrate is reduced to paste and about 5%-80% adjuvant(s) (such as calcium-containing substance(s) and/or vitamin D₃) of the total composition is/are added to the paste. The paste is then dried and pulverized by methods, such as spray drying or lyophilization, to obtain a powder extract.

In a further embodiment of the present invention, the powder extract obtained from the previous procedure is dissolved in a sufficient ethanol-containing solvent and poured into a D101 porous resin column (polystyrene-type non-polar column). Then the extract is eluted by introducing, respectively, water (10L), about 10% ethanol (10L), 50% ethanol (10L) and 80% ethanol (10L) in that order. The obtained water eluate is discarded and the ethanol-containing elute is collected, concentrated and reduced to obtain 24 g of the product.

The product is analyzed under HPLC and UV spectrometer. The result shows that the content of total coumarins and flavnoids is above 50%.

### EXAMPLE 2

A 5L container contains 400 g of Semen cuscutae, preferably in the form of powder. Later, ethanol (or other polar solvent(s)) of about 4-10 times volume of concentration from about 20% to 80%, about preferably 50% ethanol of 2800 ml as extraction solvent, is added thereto. The liquid mixture is heated to between about 50° C. and 70° C. for about 3-10 hours and then filtered after cooling. Next, the filtrate is concentrated to paste and about 5%-80% adjuvant(s) (such as calcium-containing substance(s) and/or vitamin D₃) of the total composition is/are added to the paste. The paste is afterwards dried and pulverized by methods, such as spray drying or lyophilization, to obtain a powder extract.

The Semen cuscutae extract of Example 2 may be further processed. The powder extract obtained from the previous procedure is dissolved in a sufficient ethanol-containing solvent and poured into a D101 porous resin column (polystyrene-type non-polar column). Then the extract is eluted by introducing, respectively, water (10L), about 10% ethanol (10L), 50% ethanol (10L) and 80% ethanol (10L) in that order. The obtained water eluate is discarded and the ethanol-containing elute is collected, concentrated and reduced to obtain 20 g of the product.

When the pharmaceutical composition of the present invention is being prepared, the solvent or the polar solvent used may be a single solvent, or a combination of two or more single solvents. Suitable solvents or polar solvents are, for example but not limited thereto, water, alcohols and/or ketones. The volume of the solvents is at the discretion of persons skilled in the art when practicing the present invention. The porous resin is used in the purifying and refining process that removes impurities and concentrates effective ingredients. This process is well known to persons skilled in the art. Not intended to be limited by any theory, when the elution process is being performed, the effective ingredients first are absorbed then later released with the change of the polarity of the mobile phase. Accordingly, the effective ingredients are purified and separated.

Moreover, the animal tests show that the pharmaceutical composition of the present invention, whether containing extract of Semen cuscutae only, or extract of both Semen cuscutae and Fructus cnidii, with or without calcium-containing substance(s) and/or vitamin D₃, can substantially increase the BMD level in lumbar vertebrae and the calcium and phosphorus levels in femur bone ash.

In the pharmaceutical composition containing Semen cuscutae and Fructus cnidii, the weight percentage of Fructus cindii is preferably in a range of about 20 to about 80 wt. %, more preferably in a range of about 30 to about 70 wt. % and Semen cuscutae is preferably in a range of about 20 to about 80 wt. %, more preferably in a range of about 30 to about 70 wt. %. The single extract of Semen cuscutae is also substantially effective. The pharmaceutical composition of the present invention, containing extract of Semen cuscutae or of Semen cuscutae and Fructus cnidii, may additionally contain additives, such as adjuvant and/or carrier and/or excipient, all known to persons skilled in the art. Some suitable adjuvants include starch, dextrin, glucose and/or magnesium carbonate.

The pharmaceutical composition, made from extract of one or both plants, with or without calcium-containing substance(s) and/or vitamin D₃, can be made into any conventional form for administration of common delivery formulation for medical and food use, such as extractum, pills, powder, tablets, capsules, transdermal patch, slow released agent, nasal inhaler, spray, liquid, injectable powder formulation, injectable formulation, instant formulation, powder formulation, drinks.

Adding one or more calcium-containing substances, for example calcium phosphates such as calcium phosphate monobasic, calcium phosphate dibasic, calcium phosphate dibasic and anhydrous, calcium phosphate tribasic, and calcium lactate, calcium gluconolactate, calcium ascorbate, calcium oxide, calcium carbonate and calcium pantothenate, and/or vitamin D₃ to the pharmaceutical composition may enhance the claimed efficacy of the present invention. The calcium-containing substance(s) and/or vitamin D₃ may be added in a range of about 5 to about 80 wt.% of the total composition. The above-mentioned calcium-containing substance(s) may also act as additive, such as adjuvant and/or carrier and/or excipient.

More importantly, animal tests of the pharmaceutical composition of the present invention, including in vivo tests, showed no detectable LD₅₀ value. No maximum dose is determined. No animal died when the oral administration dosage reached 20,000mg/kg.

After the animals were put to death, examination with the naked eye of the important internal organs such as the heart, the liver, the spleen, lungs, kidneys, and gastrointestinal organs showed no substantial abnormality. This suggests that, unlike the separate administration of the effective ingredient Fructus cnidii, the pharmaceutical composition of the present invention raises little concerns of oral administration toxicity.

In order to determine the efficacy against osteoporosis in rats (strain Sprague-Dawley), two animal test models were constructed to illustrate the outcomes against normal osteoporosis or estrogen-deficiency-induced osteoporosis, respectively.

In the animal test models, retinoic acid-induced osteoporosis model was used as normal osteoporosis model and ovariectomy-induced model was used as the model for osteoporosis induced by estrogen deficiency.

### EXAMPLES PART (1)

Retinoic Acid-Induced Osteoporosis Model

The following experiment was conducted to test the effects of the pharmaceutical composition of the present invention on rats with retinoic acid-induced osteoporosis in terms of bone histomorphometry and illustrate the claimed efficacy of the pharmaceutical composition.

—Materials and Resources—

The Pharmaceutical Composition of the Present Invention: Batch No. 20020934, provided by Nuliv Biomedicine Inc.

Retinoic Acid (RA): Batch No. 01006, provided by Shanghai Sixth Pharmaceuticals.

Gushukang: Kan-Chen Pharmaceuticals (contains Herba Epimedii, Radix Rehmanniae Praeparata, Radix Astragali seu Hedysari, radix Salviae Miltiorrhizae, etc.), Batch No. 000402.

Calcium-containing Substance: calcium carbonate plus adequate vitamin D₃ Solvent: 0.5% carboxymethylcellulose sodium solution, (CMC-Na, the basic solvent)

Method of Formulation: Each dosage is precisely weighed and dissolved in the basic solvent to obtain the required concentration. Each animal is administered with the dosage of 1 ml/100 g according to its body weight.

—Equipment and Devices—

SX-40 A X-ray bone densitometer, H66005; Ion COATER, IB 3, EikoCMIAS-98A Microscope Image Analysis Measuring System, Beihang University; electronic balance, BS110S, Sartorius, Germany.

—Animals—

Resource, Species, Strain and License: Sprague-Dawley, SPF rats, provided by SIPPR/BK LTD.

Age: 3 months

Sex: male

Numbers of Each Group: 8

—Experiment Procedure—

72 healthy male rats were divided into 9 groups of 8 rats. 15 days within the beginning of the experiment, rats in the Normal Group were administered every morning with the basic solvent and rats in the other groups were administered with retinoic acid with the dosage of 70 mg/kg. For five consecutive weeks since the beginning of the experiment, rats in each group were orally administered in the afternoon with the following substances:

Normal Group: CMC-Na solution 10 ml/kg

Affected Group: CMC-Na solution 10 ml/kg

Positive Group: Gushukang 3 g/kg

Experiment Groups:

Subgroup I—Extract of Semen Cuscutae and Fructus Cindii/Composite Groups

(Low Dosage): 100 mg/kg

(Medium Dosage): 200 mg/kg

(High Dosage): 400 mg/kg

Subgroup II—Sole Extract of Semen cuscutae (Unitary Group)

Dosage: 200 mg/kg

Subgroup III—Low Dosage Plus Calcium-containing Substance 100 mg/kg, 200 mg/kg in Total

Subgroup IV—Unitary Group Plus Calcium-containing Substance in a Ratio of 70/30, 200 mg/kg in Total

The BMD level, calcium level and phosphorous level in lumbar vertebrae and femur of each mouse were measured at the end of the fifth week.

The results of BMD analysis are shown in Table 1. After consecutive administration for five weeks, the BMD level in lumbar vertebrae and femurs in Affected Group are prominently lower than that of Normal Group. A successful model was therefore created. For the treated groups, after being consecutively administered for five weeks, the composite groups of Low Dosage 100 mg/kg, Medium Dosage 200 mg/kg and High Dosage 400 mg/kg as well as Unitary Group, Subgroup III and Subgroup IV, all clearly showed substantially elevated BMD level in lumbar vertebrae compared to that of the Affected Group. In terms of the change of BMD level in femurs, both Subgroups III and IV showed essentially positive results.

TABLE 1
The effects of the composite and unitary pharmaceutical
compositions of the present invention on the BMD level
in lumbar vertebrae and femurs for the retinoic acid-
induced osteoporosis (n = 6, x ± SD)

| | | Lumbar | |
|---|---|---|---|
| | | vertebrae | Femurs |
| Group | Dosage | (g/cm²) | (g/cm²) |
| Normal Group | 10 ml/kg | 0.315 ± 0.050* | 0.459 ± 0.070* |
| (normal group/treated | | | |
| with 0.5% CMC-Na) | | | |
| Affected Group | 10 ml/kg | 0.258 ± 0.018 | 0.376 ± 0.027 |
| (treated with 0.5% | | | |
| CMC-Na) | | | |
| Low Dosage | 100 mg/kg | 0.274 ± 0.029 | 0.408 ± 0.015* |
| Medium Dosage | 200 mg/kg | 0.293 ± 0.025* | 0.408 ± 0.024 |
| High Dosage | 400 mg/kg | 0.280 ± 0.006* | 0.405 ± 0.012* |
| Subgroup III | 200 mg/kg | 0.290 ± 0.015* | 0.408 ± 0.020 |
| Unitary Group | 200 mg/kg | 0.282 ± 0.017* | 0.409 ± 0.018 |
| Subgroup IV | 200 mg/kg | 0.284 ± 0.028* | 0.410 ± 0.025 |
| Positive Group | 3000 mg/kg | 0.285 ± 0.031 | 0.429 ± 0.034 |
| (Gushukang) | | | |

*p < 0.05,
**p < 0.01,
***p < 0.001, compared to Affected Group

The results of bone mineral content analysis are shown in Table 2. After consecutive administration for five weeks, there is a decrease in (bone) ash mass index (ash weight/dried weight) when Affected Group is compared with Normal Group (normal group). Nevertheless, both the calcium and the phosphorus levels in bone ash are substantially lowered. For the treated groups, i.e. the composite groups of Low Dosage 100 mg/kg, Medium Dosage 200 mg/kg and High Dosage 400 mg/kg as well as Unitary Group, Subgroup III and Subgroup IV, after consecutive administration for five weeks, a substantial rise in calcium and phosphorus levels in femurs was observed in Medium Dosage (200 mg/kg), High Dosage (400 mg/kg) and Subgroup III.

TABLE 2
The effects of the composite and unitary pharmaceutical compositions
of the present invention on the femur ash mass index of and bone
mineral content in rats with retinoic acid-induced osteoporosis

| | | ash mass | calcium level | Phosphorus level |
|---|---|---|---|---|
| Group | Dosage | index (g/g) | (g/g) | (g/g) |
| Normal Group | 10 ml/kg | 0.588 ± 0.068 | 0.142 ± 0.011* | 0.125 ± 0.013* |
| (normal bone/treated | | | | |
| with 0.5% | | | | |
| CMC-Na) | | | | |
| Affected Group | 10 ml/kg | 0.568 ± 0.020 | 0.129 ± 0.008 | 0.112 ± 0.005 |
| (treated with 0.5% | | | | |
| CMC-Na) | | | | |
| Low Dosage | 100 mg/kg | 0.576 ± 0.020 | 0.129 ± 0.031 | 0.116 ± 0.026 |
| Medium Dosage | 200 mg/kg | 0.576 ± 0.014 | 0.150 ± 0.010* | 0.133 ± 0.008*** |
| High Dosage | 400 mg/kg | 0.594 ± 0.059 | 0.151 ± 0.013** | 0.133 ± 0.013*** |
| Subgroup III | 200 mg/kg | 0.580 ± 0.025 | 0.151 ± 0.018* | 0.132 ± 0.013* |
| Unitary Group | 200 mg/kg | 0.572 ± 0.038 | 0.110 ± 0.031* | 0.100 ± 0.033*** |
| Subgroup IV | 200 mg/kg | 0.574 ± 0.054 | 0.115 ± 0.026* | 0.110 ± 0.038** |
| Positive Group | 3000 mg/kg | 0.574 ± 0.015 | 0.148 ± 0.010** | 0.128 ± 0.012** |
| (Gushukang) | | | | |

*p < 0.05,
**p < 0.01,
***p < 0.001, compared to Affected Group

Therefore, in the retinoic acid-induced osteoporosis model, substantial positive results were found in the composite pharmaceutical composition and Subgroup III of the present invention with respect to the change of calcium and phosphorous levels in bone ash. The results also suggest one advantage of the present invention—low toxicity. The results show the efficacy of the pharmaceutical composition of the present invention for the prophylaxis and treatment of osteoporosis.

The bone histomorphometry index indicated that, when compared with Normal Group, the Affected Group had lower percentage of trabecular area and lower average trabecular width (μm) in the femurs, which suggested a successful model. In High Dosage, Medium Dosage, Subgroup III as well as Unitary Group and Subgroup IV, trabecular area percentage and average trabecular width are substantially higher than those in Affected Group. The percentage of trabecular area in low dosage group is substantially raised, too. See Table 3.

TABLE 3
The effects of the composite and unitary pharmaceutical compositions
of the present invention on femur histomorphometry index for the
retinoic acid-induced osteoporosis (n = 6, x ± SD)

| | Arch Structure of | Observation of | Area % of | Average Width of |
|---|---|---|---|---|
| Group | Trabcaluae | Bone Surface | Trabcaluae | Trabcaluae (μm) |
| Normal Group | ovoid/ellipse, | more even, | 0.805 ± 0.047*** | 155.33 ± 19.38*** |
| (See Above) | smaller cage | smoother | | |
| Affected Group | ellipse/sharp | uneven, not | 0.665 ± 0.046 | 46.00 ± 10.35 |
| (See Above) | ellipse, larger cage | smooth | | |
| Low Dosage | ellipse/sharp | uneven, not | 0.785 ± 0.061* | 51.67 ± 8.71 |
| | ovoid, larger cage | smooth | | |
| Medium Dosage | ovoid/ellipse, | more even, | 0.861 ± 0.061** | 72.67 ± 9.18* |
| | smaller cage | smoother | | |
| High Dosage | ovoid/ellipse, | more even, | 0.846 ± 0.022*** | 89.33 ± 9.44*** |
| | smaller cage | smoother | | |
| Subgroup III | ovoid/ellipse, | more even, | 0.881 ± 0.084** | 75.87 ± 9.25* |
| | smaller cage | smoother | | |
| Unitary Group | ovoid/ellipse/sharp | a little more | 0.796 ± 0.055** | 61.00 ± 11.01* |
| | ellipse, larger cage | even, a little | | |
| | | smoother | | |
| Subgroup IV | ovoid/ellipse/sharp | a little more | 0.806 ± 0.087** | 62.05 ± 15.05* |
| | ellipse, larger cage | even, a little | | |
| | | smoother | | |
| Positive Group | ovoid/ellipse, | more even, | 0.854 ± 0.033** | 104.67 ± 9.69*** |
| (See Above) | smaller cage | smoother | | |

*p < 0.05,
** p < 0.01,
*** p < 0.001, compared to Affected Group

In High Dosage Group and Subgroup III, the composite pharmaceutical composition of the present invention helps increase bone mineral density and improve trabecular structure with respect to the retinoic acid-induced osteoporosis. Low Dosage Group, Unitary Group and Subgroup WV also show certain degree of improvement with respect to the retinoic acid-induced osteoporosis.

### EXAMPLES PART (II)

Ovariectomy-Induced Osteoporosis Model

The following experiment was conducted to test the effects of the pharmaceutical composition of the present invention on rats with ovariectomy-induced osteoporosis in terms of bone mineral contents, bone mineral density, biomechanical index and bone histomorphometry as well as illustrate the claimed efficacy of the pharmaceutical composition.

The ovariectomized animal model was constructed as follows. Anesthesia of the rats was conducted by intra-abdominal injection of 20% urethane according to the dosage of 1 g/kg. The rats were then placed face down, incised in both sides of the spinal cord under aseptic condition, and their ovaries were removed to attain an osteoporosis animal model. For Normal Group, a piece of fat was removed on both sides of back spinal cord. After five days, 72 animals were chosen from the living rats and randomly divided into 9 groups of 8 animals. Administrations were given according to the following conditions:

Normal Group (pseudo-operation): CMC-Na solution 10 ml/kg/d

Affected Group: CMC-Na solution 10 ml/kg/d

Positive Group (Nylestriol): batch no. 020701, provided by Shianghai Hualian Pharmaceuticals, 1 mg/kg, once a week

Experiment Groups:

Subgroup I—Extract of Semen Cuscutae and Fructus Cindii/Composite Groups

(Low Dosage): 100 mg/kg

(Medium Dosage): 200 mg/kg

(High Dosage): 400 mg/kg

Subgroup II—Sole Extract of Semen Cuscutae (Unitary Group)

Dosage: 200 mg/kg

Subgroup III—Low Dosage Plus Calcium-containing Substance 100 mg/kg, 200 mg/kg in Total

Subgroup IV—Unitary Group Plus Calcium-containing Substance in a Ratio of 70/30, 200 mg/kg in Total

The animals were continuously administered for 3 months. The effects of the pharmaceutical composition of the present invention on morphological alteration of the femur of rats were analyzed using a Dual Energy X-Ray Absorptiometry (DEXA) two days before end of the experiment under the anesthesia of ether. The femur and tibia bone were peeled and measured. Those pieces were soaked in 4% glutaraldehyde (0.1M, pH 7.3) for more than 24 hours. Later on, the vector side of the femur was hacksawed with dental diamond saw blades (grindstone). One piece from each animal was removed and cleaned in 10% sodium hypochlorite solvent for 6 hours, followed by ultra sound wash for 15 minutes.

It was then dehydrated by ethanol-gradient, soaked in ethyl ether, air dried, and then treated with ion sputtering coating (IB-3). The BMD was assessed by dual-energy X-ray absorptiometry (DEXA), and the SX-40 X-ray bone densitometer on 20,000 volt. The graphs were taken by CMIAS-98A image analyzer.

The bones were dried at 10° C. in the oven and then weighed to get the dry weight. The dry bones were carbonated and then incinerated in 800° C. muffle furnace for 6 hours. The ashes were weighed and then dissolved in 6N HCl, in order to measure the Ca and P levels.

In the Affected Group, the (bone) ash mass index (ash weight/dry weight) was substantially lowered. The calcium and phosphorous levels were lowered but the decrease was not statistically significant. In the composite High Dosage Group, Medium Dosage Group and Subgroup III, ash mass index values were substantially higher. See Table 4.

TABLE 4
The effects of the composite and unitary pharmaceutical
compositions of the present invention on ash mass index
and bone mineral content in rats with ovariectomy-
induced osteoporosis (n = 8, x ± SD)

| Group | Ash Mass Index (g/g) | Ca level (g/g) | P level (g/g) |
|---|---|---|---|
| Normal Group | 0.667 ± 0.029** | 0.261 ± 0.029 | 0.133 ± 0.024 |
| Affected Group | 0.619 ± 0.030 | 0.243 ± 0.029 | 0.123 ± 0.007 |
| Positive Group | 0.649 ± 0.024* | 0.254 ± 0.029 | 0.130 ± 0.014 |
| Unitary Group | 0.627 ± 0.055 | 0.242 ± 0.019 | 0.131 ± 0.014 |
| Subgroup IV | 0.630 ± 0.065 | 0.245 ± 0.026 | 0.135 ± 0.028 |
| Low Dosage | 0.647 ± 0.055 | 0.243 ± 0.030 | 0.123 ± 0.015 |
| Medium | 0.665 ± 0.035* | 0.258 ± 0.019 | 0.126 ± 0.009 |
| Dosage | | | |
| High Dosage | 0.659 ± 0.028* | 0.255 ± 0.034 | 0.132 ± 0.013 |
| Subgroup III | 0.666 ± 0.056* | 0.259 ± 0.020 | 0.128 ± 0.018 |

*p < 0.05,
**p < 0.01,
***p < 0.001, compared to Affected Group

The DEXA analysis results showed that the BMD level in lumbar spine and femoral head in Affected Group were substantially lower. In Subgroup I, Subgroup III as well as Unitary Group and Subgroup IV, the BMD level in femoral head was substantially higher. Medium Dosage Group showed substantially higher bone mineral density in lumbar vertebrae. See Table 5.

TABLE 5
The effects of the composite and unitary pharmaceutical
compositions of the present invention on BMD levels in
lumbar spine and femoral head of rats with ovariectomy-
induced osteoporosis (n = 6, x ± SD)

| | | Lumbar | |
|---|---|---|---|
| Group | Dosage | vertebra (g/cm²) | Femur (g/cm²) |
| Normal Group | 10 ml/kg | 0.299 ± 0.032* | 0.404 ± 0.051** |
| Affected Group | 10 ml/kg | 0.261 ± 0.015 | 0.304 ± 0.033 |
| Positive Group | 1 mg/kg/w | 0.263 ± 0.015 | 0.353 ± 0.030* |
| Unitary Group | 200 mg/kg/d | 0.284 ± 0.027 | 0.348 ± 0.023* |
| Subgroup IV | 200 mg/kg/d | 0.288 ± 0.037 | 0.349 ± 0.038* |
| Low Dosage | 100 mg/kg/d | 0.281 ± 0.033 | 0.362 ± 0.030* |
| Medium Dosage | 200 mg/kg/d | 0.295 ± 0.031* | 0.356 ± 0.041* |
| High Dosage | 400 mg/kg/d | 0.266 ± 0.029 | 0.348 ± 0.015* |
| Subgroup III | 200 mg/kg/d | 0.297 ± 0.038* | 0.342 ± 0.048* |

*p < 0.05,
**p < 0.01,
***p < 0.001, compared to Affected Group

Biomechanical Index

Please see Table 6. The results showed that in Affected Group, the maximum torsion index δb(mm), the maximum strength Pb(N) and tensile strength δb(MPa) were significantly lower. The spring strength K(N/mm) and the cross-sectional spring coefficient Wx(mm3) were substantially higher. The δb(MPa) value in all High Dosage, Medium Dosage, Subgroup III as well as Unitary Group and Subgroup IV was significantly higher than that of Affected Group. The Wx(mm3) value in High Dosage was significantly lower. In terms of EI(KNmm²), there was no significant difference among the groups.

TABLE 6
The effects of the composite and unitary pharmaceutical
compositions of the present invention on biomechanical index
of rats with ovariectomy-induced osteoporosis (n = 8, x ± SD)

| Group | δ_{b}(mm) | P_{b}(N) | σ_{b}(MPa) | K (N/mm) | EI(KN · mm²) |
|---|---|---|---|---|---|
| Normal Group | 0.73 ± 0.23 | 110 ± 14.3 | 154.09 ± 20.25* | 253.03 ± 69.38 | 39.98 ± 15.11 |
| Affected Group | 0.54 ± 0.23 | 105 ± 4.5 | 131.19 ± 12.57 | 289.59 ± 55.35 | 37.93 ± 10.28 |
| Positive Group | 0.54 ± 0.17 | 97 ± 9.4* | 147.79 ± 13.95* | 248.91 ± 53.04 | 28.55 ± 7.03 |
| Unitary Group | 0.81 ± 0.25* | 100 ± 12.7 | 167.83 ± 29.44** | 219.96 ± 18.59** | 36.66 ± 3.10 |
| Subgroup IV | 0.79 ± 0.21* | 103 ± 15.6 | 165.58 ± 26.56* | 220.57 ± 28.43* | 36.89 ± 5.23 |
| Low Dosage | 0.58 ± 0.18 | 103 ± 21.4 | 133.40 ± 22.86 | 264.34 ± 56.38 | 35.71 ± 9.04 |
| Medium | 0.61 ± 0.21 | 117 ± 17.5 | 162.01 ± 18.01** | 277.41 ± 78.40 | 37.04 ± 11.43 |
| Dosage | | | | | |
| High Dosage | 0.68 ± 0.30 | 107 ± 20.2 | 162.00 ± 24.83** | 262.50 ± 91.83 | 34.06 ± 8.21 |
| Subgroup III | 0.63 ± 0.32 | 119 ± 19.5 | 160.11 ± 19.01** | 279.62 ± 55.42 | 38.15 ± 10.43 |

*p < 0.05,
**p < 0.01,
***p < 0.001, compared to Affected Group

The bone histomorphometry index indicated that, when compared with Normal Group, the Affected Group had lower percentage of trabecular area and lower average trabecular width (μm) in the femurs. In both Subgroup I and Subgroup III, the percentage of trabecular area and the average trabecular width were substantially higher. Also, the average trabecular width in both Unitary Group and Subgroup IV were significantly higher than those in Affected Group. See Table 7.

TABLE 7
The effects of the composite and unitary pharmaceutical compositions
of the present invention on the femur histomorphometry index in rats
with ovariectomy-induced osteoporosis (n = 6, x ± SD)

| | | | | | Average |
|---|---|---|---|---|---|
| | | | Bone | Percentage of | Trabecular Width |
| Group | Dosage | Trabecular Structure | Surface | Trabecular Area | (μm) |
| Normal | 10 ml/kg | round/ovoid/ellipse, | more even, | 0.9196 ± 0.0402*** | 133.00 ± 24.71*** |
| Group | | smaller cage | smoother | | |
| Affected | 10 ml/kg | ellipse, larger cage | uneven, less | 0.7978 ± 0.0409 | 57.67 ± 19.78 |
| Group | | | smoother | | |
| Positive | 1 mg/kg/w | ovoid/ellipse, | more even, | 0.9148 ± 0.0371** | 146.89 ± 22.27*** |
| Group | | smaller cage | smoother | | |
| Unitary | 200 mg/kg/d | sharp ovoid/sharp | uneven, less | 0.8301 ± 0.0445 | 116.83 ± 5.49*** |
| Group | | ellipse, smaller cage | smoother | | |
| Sub- | 200 mg/kg/d | sharp ovoid/sharp | uneven, less | 0.8505 ± 0.0478 | 115.58 ± 6.68** |
| group IV | | ellipse, smaller cage | smoother | | |
| Low | 100 mg/kg/d | ellipse/sharp ellipse, | uneven, less | 0.8829 ± 0.0487** | 99.67 ± 14.00** |
| Dosage | | larger cage | smoother | | |
| Medium | 200 mg/kg/d | ovoid/ellipse, | more even, | 0.9244 ± 0.0317*** | 130.00 ± 27.42*** |
| Dosage | | smaller cage | smoother | | |
| High | 400 mg/kg/d | ovoid/ellipse, | more even, | 0.9493 ± 0.0196*** | 142.33 ± 29.62*** |
| Dosage | | smaller cage | smoother | | |
| Sub- | 200 mg/kg/d | ovoid/ellipse, | more even, | 0.934 ± 0.0027** | 135.06 ± 30.63** |
| group III | | smaller cage | smoother | | |

*p < 0.05,
**p < 0.01,
***p < 0.001, compared to Affected Group

The experiment results suggested that the composite and the unitary pharmaceutical compositions as well as Subgroups III and IV of the present invention reduced bone conversion, increased bone mineral density and enhanced the trabecular structure. Also, in these groups, the ash mass index was substantially lower and Ca and P levels were higher. Ash mass index was substantially increased in all High Dosage, Medium Dosage and Subgroup III and Ca and P levels were increased by a certain degree, too. It was therefore concluded that the pharmaceutical compositions as well as those with calcium-containing substances of the present invention could increase the bone mineral content in femurs, enhance the hardness of bones, reduce the risk of fracture and promote bone health. The experiment also indicated that indexes such as tensile strength δb were decreased in the Affected Group but increased in all Subgroups I, II, III and IV, which illustrated that many aspects are improved therefore when compared to the Affected Group. After administration for 3 months, all Subgroups I, II, III and IV showed improved results in trabecular structure, bone surface, the percentage of trabecular area and average trabecular width, which illustrated that the pharmaceutical composition of the present invention could substantially improve the bone structure in many aspects.

Oral Acute Toxicity of the Pharmaceutical Composition

20 Kuanmin house mice, 10 males and 10 females, underwent fasting for 12 hours and were then administered with the pharmaceutical composition of the present invention under the dosage of 0.8 ml/20 g (maximum administration dosage). They were then monitored for 14 days. Afterwards, the internal organs were taken out for examination. The results showed that after the oral administration, the behavior of the mice was back to normal in 2 hours. In additional, autopsy indicated that the important internal organs such as the heart, the liver, the spleen, lungs, kidneys and gastrointestinal organs did not show substantial abnormality to the naked eye. Because the maximum oral dose was not determined at the dosage of 20,000 mg/kg, it was therefore concluded that the maximum tolerance must exceed 20,000 mg/kg.

Maximum Tolerance of Abdominal Injection of the Pharmaceutical Composition

20 Kuanmin house mice, 10 males and 10 females, underwent fasting for 12 hours and were abdominally injected with the pharmaceutical composition of the present invention at the dosage of 0.8 ml/20 g (50 mg/ml as the maximum administration dosage). They were monitored for 14 days. Afterwards, the internal organs were again taken out for examination. The results showed that after the injection, the behavior of the mice showed no abnormality. In addition, autopsy results indicated that the important internal organs such as such as the heart, the liver, the spleen, lungs, kidneys and gastrointestinal organs did not show substantial abnormality to the naked eye. Because the maximum abdominal injection dosage was not determined at the dosage of 2,000 mg/kg, it is therefore concluded that the maximum tolerance must exceed 2,000 mg/kg.

By means of the detailed descriptions of what is presently considered to be the most practical and preferred embodiments of the subject invention, it is the expectation that the features and the gist thereof are plainly revealed. Nevertheless, these above-mentioned illustrations are not intended to be construed in a limiting sense. Instead, it should be well understood that any analogous variation and equivalent arrangement is supposed to be covered within the spirit and scope to be protected and that the interpretation of the scope of the subject invention would therefore as much as broadly apply.

We claim:

1. A pharmaceutical composition for prophylaxis and treatment of osteoporosis, comprising an extract of a mixture of Fructus cindii and Semen cuscutae, and one or more pharmacologically acceptable adjuvant and/or carrier and/or excipient.

2. The pharmaceutical composition of claim 1, wherein said extract is obtained by (1) extraction of said mixture by ethanol or other polar solvent(s), (2) filtration, and (3) concentration.

3. The pharmaceutical composition of claim 1, wherein Fructus cindii is in a range of about 20 to about 80 wt. %.

4. The pharmaceutical composition of claim 3, wherein Fructus cindii is in a range of about 30 to about 70 wt. %.

5. The pharmaceutical composition of claim 1, wherein Semen cuscutae is in a range of about 20 to about 80 wt. %.

6. The pharmaceutical composition of claim 5, wherein Semen cuscutae is in a range of about 30 to about 70 wt. %.

7. The pharmaceutical composition of claim 1, in the form of extractum, pills, powder, tablets, capsules, transdermal patch, slow released agent, nasal inhaler, spray, liquid, injectable formulation, instant formulation, powder formulation or drinks.

8. A pharmaceutical composition for prophylaxis or treatment of osteoporosis, comprising a mixed extract of Fructus cindii and Semen cuscutae, and one or more pharmacologically acceptable additive(s).

9. The pharmaceutical composition of claim 8, wherein said mixed extract is obtained by (1) extraction of a mixture of Fructus cindii and Semen cuscutae by an extraction solvent, (2) filtration, and (3) concentration.

10. The pharmaceutical composition of claim 8, wherein Fructus cindii is in a range of about 20 to about 80 wt. %.

11. The pharmaceutical composition of claim 10, wherein Fructus cindii is in a range of about 30 to about 70 wt. %.

12. The pharmaceutical composition of claim 8, wherein Semen cuscutae is in a range of about 20 to about 80 wt. %.

13. The pharmaceutical composition of claim 12, wherein Semen cuscutae is in a range of about 30 to about 70 wt. %.

14. The pharmaceutical composition of claim 8, further comprising one or more calcium-containing substance(s) and/or vitamin D₃.

15. The pharmaceutical composition of claim 14, wherein said calcium-containing substance is selected from the group consisting of calcium phosphate (monobasic, dibasic, dibasic and anhydrousor tribasic), calcium lactate, calcium gluconolactate, calcium ascorbate, calcium oxide, calcium carbonate and calcium pantothenate.

16. The pharmaceutical composition of claim 8, in the form of extractum, pills, powder, tablets, capsules, transdermal patch, slow released agent, nasal inhaler, spray, liquid, injectable formulation, instant formulation, powder formulation or drinks.

17. A pharmaceutical composition for prophylaxis or treatment of osteoporosis, comprising an extract of Semen cuscutae, and one or more pharmacologically acceptable additive(s).

18. The pharmaceutical composition of claim 17, wherein said extract is obtained by (1) extraction of Semen cuscutae by an extraction solvent, (2) filtration, and (3) concentration.

19. The pharmaceutical composition of claim 17, further comprising one or more calcium-containing substance(s) and/or vitamin D₃.

20. The pharmaceutical composition of claim 19, wherein said calcium-containing substance is selected from the group consisting of calcium phosphate(monobasic, dibasic, dibasic and anhydrous, or tribasic), calcium lactate, calcium gluconolactate, calcium ascorbate, calcium oxide, calcium carbonate and calcium pantothenate.

21. The pharmaceutical composition of claim 17, in the form of extractum, pills, powder, tablets, capsules, transdermal patch, slow released agent, nasal inhaler, spray, liquid, injectable formulation, instant formulation, powder formulation or drinks.

22. A method for preparing a pharmaceutical composition for osteoporosis, comprising the steps of (a) mixing a first quantity of Fructus cindii with a second quantity of Semen cuscutae to form a mixture;
(b) adding sufficient ethanol of concentration of about 20% to about 80% into said mixture then heating;
(c) filtering said mixture after said mixture cools down;
(d) drying and pulverizing said mixture to obtain an extract;
(e) dissolving said extract in a sufficient solvent and pouring said extract into a prepared porous resin column; and
(f) performing elution by in succession adding solutions of different concentration of ethanol and water as eluant into said prepared porous resin column to obtain said pharmaceutical composition.

23. A method for preparing a pharmaceutical composition for osteoporosis, comprising the steps of:
(a) providing a material comprising Semen cuscutae;
(b) adding an sufficient extraction solvent into said material to obtain an extraction mixture then leaving said extraction mixture under heating;
(c) filtering said extraction mixture after said extraction mixture cools down;
(d) drying and pulverizing said extraction mixture to obtain an extract;
(e) dissolving said extract in a sufficient solvent and pouring said extract into a prepared porous resin column; and
(f) performing elution by adding solutions of different concentration of ethanol and water as eluant into said prepared porous resin column to obtain said pharmaceutical composition.

24. The method of claim 23, wherein said material further comprises Fructus cnidii.

25. The method of claim 23, wherein said porous resin column is a non-polar column.

26. The method of claim 23, wherein said extraction solvent comprises ethanol.
